(19) United States
(12) Patent Application Publication
(10) Pub. No.: US 2025/0281777 A1
(43) Pub. Date: Sep. 11, 2025

(54) VENTILATED PROTECTION DEVICE FOR THE RESPIRATORY TRACT

(71) Applicant: Silvio Marino, Albiano Magra-Aulla (MS) (IT)

(72) Inventor: Silvio Marino, Albiano Magra-Aulla (MS) (IT)

(21) Appl. No.: 18/859,662

(22) PCT Filed: Apr. 13, 2023
(86) PCT No.: PCT/EP2023/025173

§ 371 Date: Oct. 24, 2024

(30) Foreign Application Priority Data
May 10, 2022 (IT) 102022000009635

(51) Int. Cl. A62B 18/00; A61M 16/06; A61M 16/10; A61M 21/00; A61M 21/02; A62B 18/10; A62B 23/02

(52) U.S. Cl. CPC A62B 18/006; A61M 16/06; A61M 16/105; A61M 21/02; A62B 18/10; A62B 23/02; A61M 2021/0016

(57) ABSTRACT

A protection device for the protection of the respiratory tract of a person includes a mask body {1} covering a person's mouth and nose, a pair of housings on sides of the mask body to hold a sleeve inside each, a spring inside each sleeve, having a first end and a second end with a fixing arrangement enabling the ends thereof to join together behind a person's head, each spring coated at a first portion fixed in the sleeve by a non-breathable fabric cylinder and at a second portion, by a breathable fabric cylinder, the front part of the mask body including a removable housing box containing a fan which facilitates, by a connection hole, recirculation of air inside the mask body, which is sucked by one sleeve through a connection tube between them and the housing box, by a non-return valve in the lower end of the respective sleeve.

This invention refers to a respiratory tract protection device equipped with a breathing aid by fan.

Various devices for protecting the airways from fine dust, fumes and liquid mists (aerosols) are commonly known. Aerosols and fine dust particles are some of the most hazardous health risks, as unfortunately we have been able to verify with the spread of the well-known Covid-19 virus, being almost invisible in breathable air. Filter masks against these particles offer protection from these dangers and are divided into three protection classes: FFP1, FFP2 and FFP3.

These masks are, moreover, prescribed in the workplace where the so-called occupational exposure limit or OEL value is exceeded. This indicates the maximum permissible concentration of dust, smoke or aerosols in the breathing air, which does not cause harm to health. When this value is exceeded, the use of filter masks becomes mandatory.

With regard to surgical masks, these have the purpose of preventing the wearer from contaminating the environment, as they limit the transmission of infectious agents and fall within the scope of medical devices referred to in Legislative Decree 24 Feb. 1997, no. 46 and successive modifications. They are used in hospitals and in places where assistance is provided to patients (for example, care homes, clinics, etc.) and, to be considered safe, they must be produced in compliance with the technical standard UNI EN 14683:2019 which provides features and test methods to evaluate resistance to liquid splashes, breathability, bacterial filtration efficiency, etc.

FFP1 masks have a reduced filtering capacity from the outside towards the wearer, of about 20%, mainly due to poor adherence to the face.

Traditional masks, however, take the air from the front and this is particularly disadvantageous as dust, fumes, aerosols are normally concentrated in the front part of the face as the operator is normally turned towards the work area and this also happens when two or more people talk to each other.

Another drawback is the fact that inside the mask there is poor air circulation which can have negative consequences for the person's health.

The inventor of the present invention has already filed, on 15/10/2020, the Patent application for Industrial Invention No. 102020000024334 “Protection device for the respiratory tract”, not yet granted, in order to provide a protection device for the respiratory tract that is more efficacious than the existing ones in terms of protection from health-damaging agents and, at the same time, is able to improve the quality of air taken from outside.

With the present invention, the applicant intends to provide a respiratory protection device which is capable of increasing ventilation inside the device itself and of diffusing inside it perfumed, balsamic or medicinal air-soluble substances.

A respiratory protection device is therefore provided according to the characteristics of the attached claim 1.

The invention will be better explained through the following description which shows the preferred embodiments made with the aid of the attached figures which respectively illustrate:

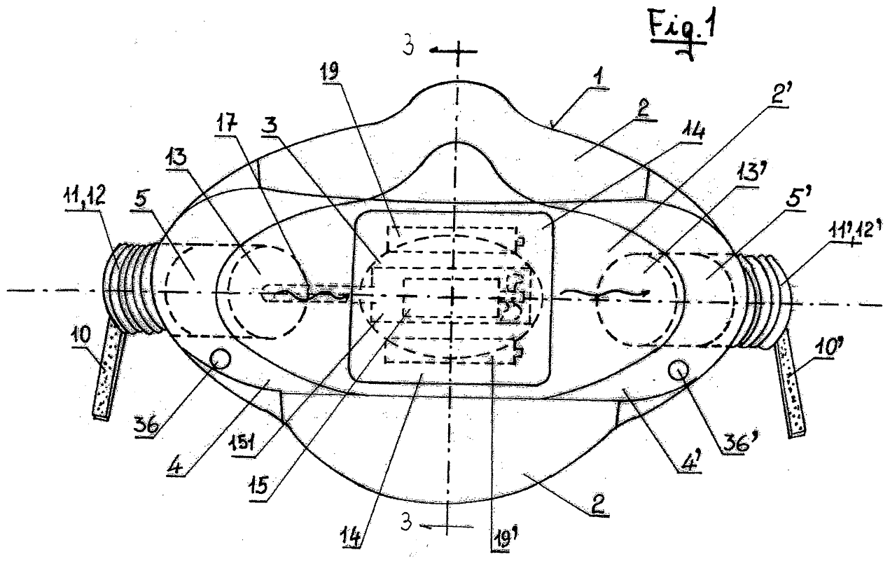

FIG. 1 illustrates a front view from the outside of the device according to this invention;

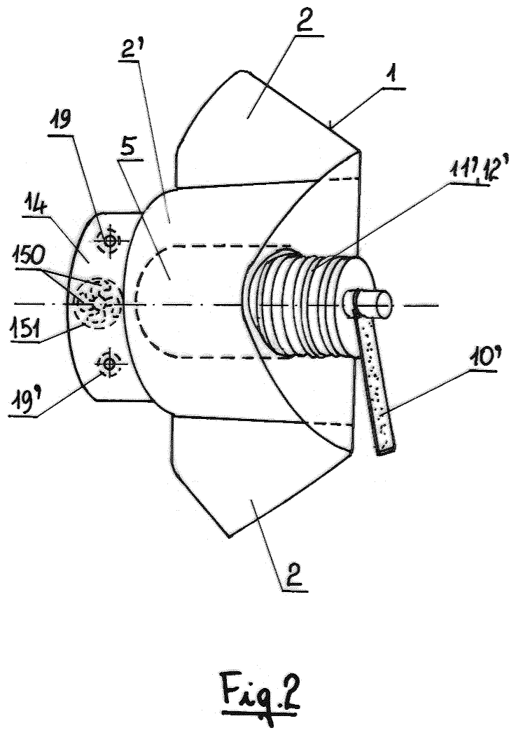

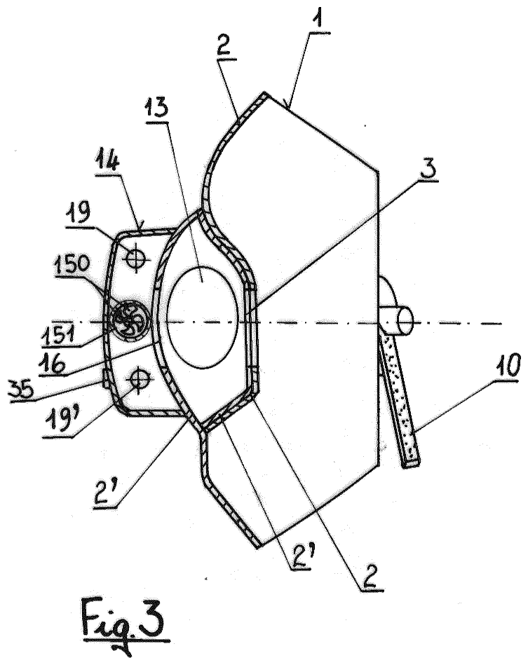

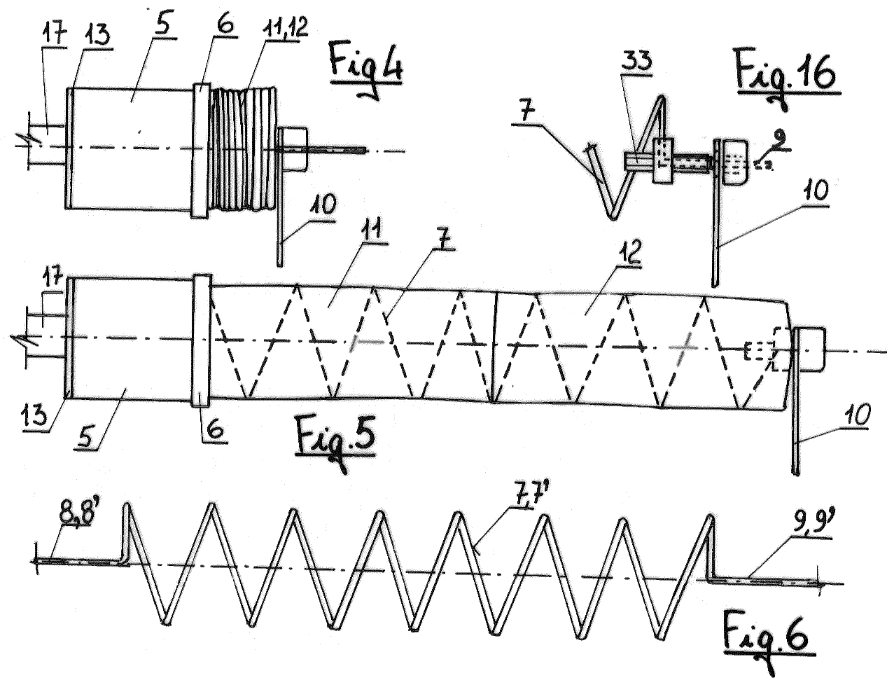

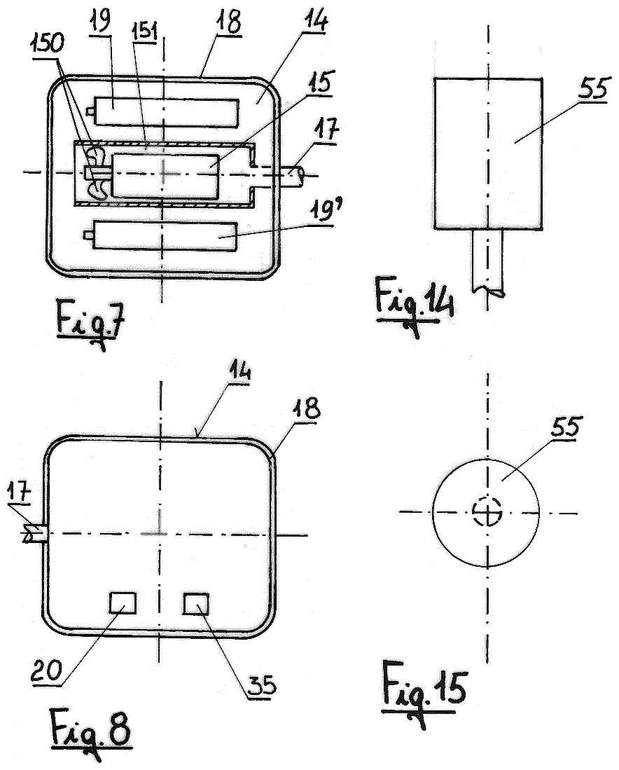

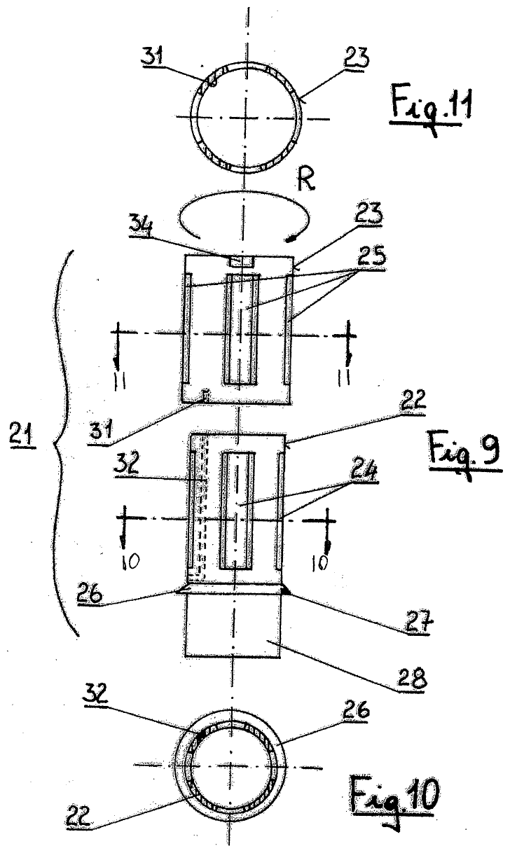

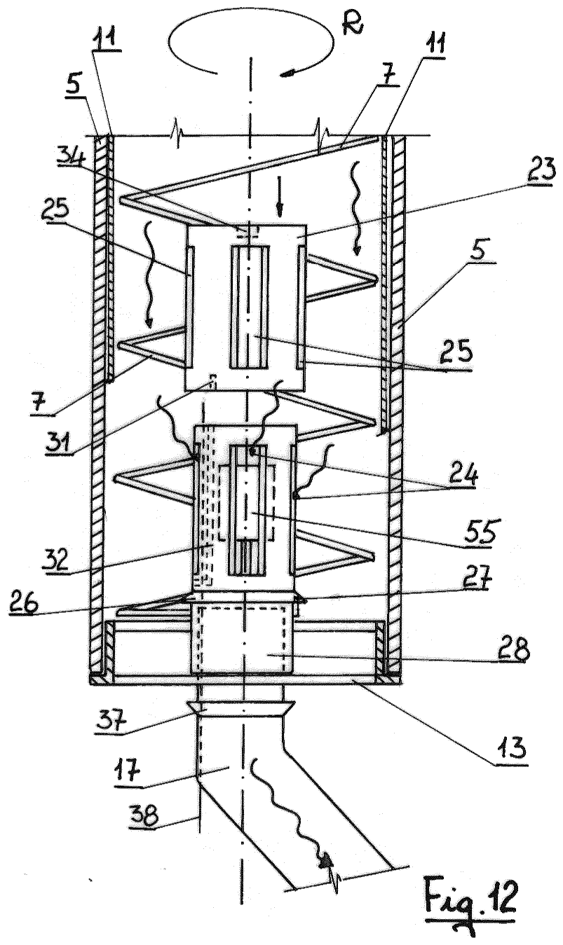

FIG. 2 illustrates a side view of the device of FIG. 1;

FIG. 3 illustrates a section according C-C of FIG. 1;

FIG. 4 illustrates the sleeve with the spring in the rest position;

FIG. 5 illustrates the sleeve with the spring in the extended position;

FIG. 6 illustrates the spring in the extended position;

FIG. 7 illustrates the inside front view and partial section of the fan housing box and relative power supply battery;

FIG. 8 illustrates the external front view of the box of FIG. 7;

FIG. 9 illustrates an exploded view of the container of the pad in the open position;

FIG. 10 illustrates the section according to A-A of FIG. 9;

FIG. 11 illustrates the section according to B-B of FIG. 9;

FIG. 12 illustrates the partial section of the sleeve with the container of the pad in the open position;

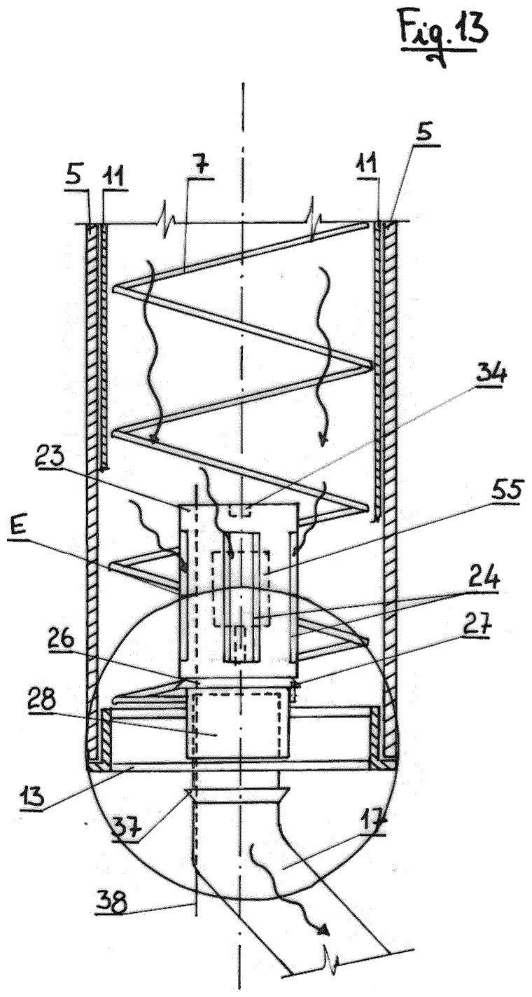

FIG. 13 illustrates the partial section of the sleeve with the container of the pad in the closed position;

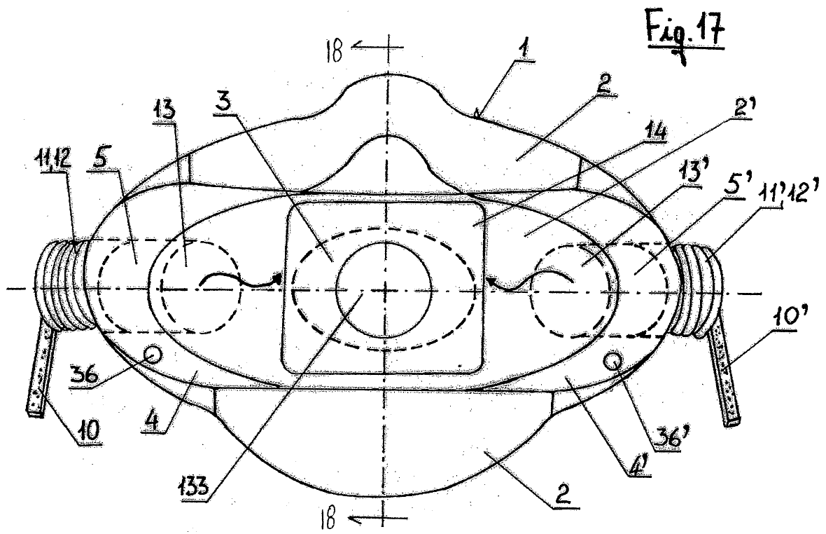

FIG. 14 illustrates the side view of the pad according to the present invention;

FIG. 15 illustrates the top view of the pad of FIG. 14;

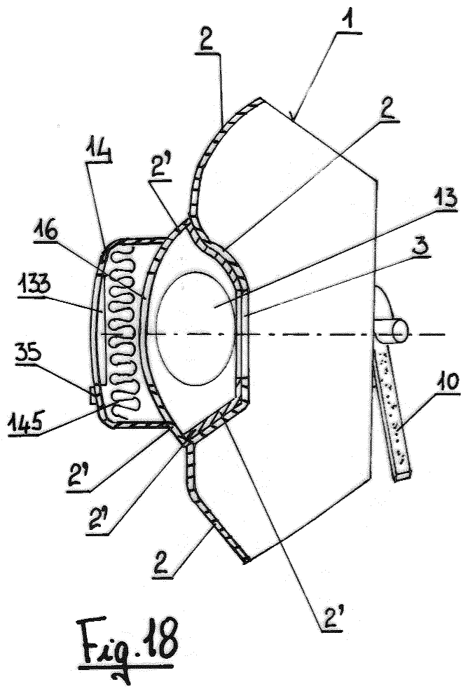

FIG. 16 illustrates the side view of the pad container closure device;

FIG. 17 illustrates a front view from the outside of an alternative embodiment of the respiratory protective device;

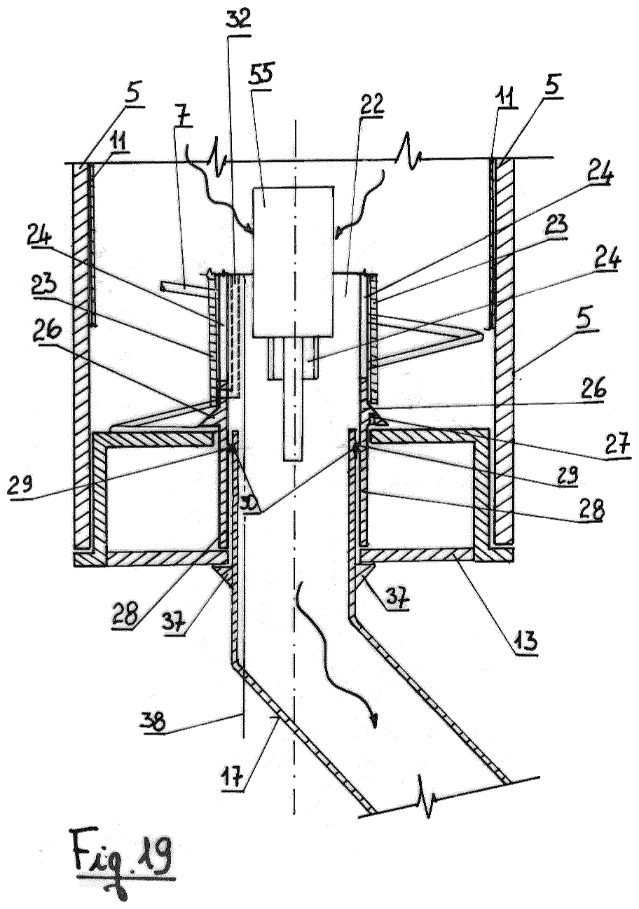

FIG. 18 illustrates the section according to D-D of FIG. 17;

FIG. 19 illustrates the enlarged detail E of FIG. 13.

With reference to the aforementioned figures, the device according to the present invention is made substantially in the form of a mask and comprises a mask body 1 of any convenient rigid or semi-rigid, soft material, suitable to cover the mouth and nose of a person, preferably formed by a first layer 2 and a second layer 2′ substantially superimposed on each other and communicating through at least one hole 3 so as to ensure the passage of air between them. At the sides of the mask body there are at least two housings 4, 4′ (one on each side) each designed to hold a respective sleeve 5 or 5′, preferably shaped like a cylinder or truncated cone and equipped with an edge 6 in the upper part so as to be able to be joined to said body 1. Said sleeve is advantageously made of a rigid or semi-rigid material.

Each sleeve 5, 5′ is provided with at least one pair of fins or other fixing means so as to make it removable from the housing 4, 4′.

Inside each sleeve 5 or 5′ a spring 7 or 7′ is inserted, preferably in metal or plastic material, provided with a first end 8 or 8′ and with a second end 9 or 9′ which has fixing means which allow the ends 9, 9′ of the two springs to join each other. Such means comprise, for example, a Velcro 10 or 10′ and the springs 7 or 7′ are capable of extending by several centimeters so as to be hooked each other by means of said Velcro 10 and 10′ behind the head of the person wearing the mask.

Each spring 7 or 7′ is coated substantially for a first portion (for example, for half of its length) by a cylinder 11 or 11′ of non-breathable fabric material and for a second portion by a cylinder 12 or 12′ of material in breathable fabric, for example, of non-woven fabric or of material of the type used for FFP1, FFP2 or FFP3 masks or other convenient material capable of filtering the air. Therefore, aeration channels connected to said mask 1 are formed.

In this way, air of higher hygienic quality is introduced, being captured by the back of the person's head and, therefore, less exposed to the dust concentrations and aerosols found in the part in front of the person's face. In addition to this, the wear and tear of the filtering material is reduced with obvious advantages in terms of economics and device reliability.

The air thus obtained is filtered by the material of said cylinder 12 or 12′ and circulates freely inside the mask 1.

In an alternative solution, all the cylinders 11, 11′, 12 and 12′ of the sleeve 5 or 5′ are made of breathable material.

Advantageously, a non-return valve 13 or 13′ is used, placed in the lower end part of the sleeve 5 or 5′, so that the air expelled in the exhalation phase, rich in carbon dioxide, does not remain inside the mask thus allowing the person wearing it to always breathe well-oxygenated air.

In this way, one of the two valves 13 or 13′ allows the entry of filtered air, while the other 13′ or 13 allows the expelled air to escape in the exhalation phase.

Advantageously, the device, according to the present invention, is equipped with a removable housing box 14, placed in the front part of the mask body 1 and containing a fan 15 which promotes, through a connection hole 16 (FIG. 3), the recirculation of the air inside the mask body 1 helping the breathing of the person. The fan 15 includes blades 150 shaped so as to draw inlet air into the mask body 1 from the non-return valve 13 or 13′ through a connection tube 17 and promote the escape of the carbon dioxide-rich air from the valve 13′ or 13. The fan 15 can be placed inside a cylindrical body 151 joined to the connection tube 17 so as to better convey the incoming air into the mask body 1.

To ensure insulation from the outside, said housing box 14 is equipped with a plastic gasket 18 and is joined to the mask body 1 by means of a snap closure. Said fan 15 operates by means of batteries 19, 19′ rechargeable preferably through wireless technology and it can be operated by means of an on/off button 20 placed at the front of said box 14.

The sleeve 5 or 5′ is internally equipped, in the central part, with a container 21 which can advantageously have a pad 55. Said container 21 is formed by two male 22 and female 23 parts overlapping each other and provided with side openings 24, 25. The male part 22, to the walls of which the pad 55 is fixed, has an enlarged base 26 preferably in the shape of a truncated cone equipped with a housing seat 27 for the first end of the spring 7 and with an appendix 28 that fits inside the central part of the non-return valve 13. Said spring 7 placed in the sleeve 5′ or 5 without the container 21, is fixed to the lower edge of the sleeve itself by means of suitable fastening means.

Said appendix 28 is also internally provided with at least one protuberance 29 able to fit into a corresponding cavity 30, as shown in FIG. 19, made at the end part of the connection tube 17 which allows the passage of air between the sleeve 5 or 5′ and the housing box 14 of the fan 15.

The connection tube 17 has also an enlarged base 37 preferably in a shape of a truncated cone which, when the connection tube 17 is inserted inside said appendix 28, abuts against the external part of the non-return valve 13 blocking it in position.

The container 21 can be closed or opened by rotation R of the female part 23 on the male part 22: in the closed position, the walls of the female part 23 close the respective openings 24 of the male part 22, while in the open position, the respective openings of the two male 22 and female 23 parts overlap each other allowing the passage of air circulating inside the sleeve 5. In order to position and block the aforementioned male and female parts, at least one tooth 31 is provided, on the inner wall of the latter, which fits inside an “L”-shaped groove 32, made on the outer wall of the male part 22 so that, once the tooth 31 has reached the end of the vertical stroke, the female part 23 locks onto the male part 22 by rotation R, remaining in position.

The rotation movement R of the female part on the male is facilitated by the presence of a pin 33 preferably hexagonal (FIG. 16) which fits into a specially shaped housing 34 (FIGS. 9, 12 and 13), preferably hexagonal in shape, placed on the top of the female part 23. A fixing element, for example a Velcro 10, is attached to said pin 33, capable of fixing said sleeve 5, 5′ behind the person's head and has inside it a housing seat for the end part of the spring.

In said way, the filtered incoming air is able to reach, through said openings 24, 25, the pad 55 which can be soaked in an aromatic, balsamic or medicinal substance and is able to release it as it passes, so that the aromatized/medicated air is conveyed inside the connection tube 17 to the housing box 14 of the fan 15 and, therefore, inside the mask body 1.

Said pad 55 is preferably activated by heating a resistance 38 placed in the immediate vicinity and/or in contact with it and it can be activated by means of an electro-thermal command 35 placed in the front part of the housing box 14.

In an alternative embodiment, the respiratory protective device provides for air inlet into the mask body 1 from both sleeves 5, 5′ and their respective non-return valves 13, 13′ while the air outlet takes place via a non-return valve 133 placed in the front of the housing box 14 and a filter 145 made of FFP2, FFP3 material.

Finally, housings 36, 36′ placed in the front part of the mask body 1 specially shaped to accommodate bluetooth earphones are provided.

The embodiments described in the present description and the configurations shown in the drawings are only the preferred embodiments of the present invention but the technical variants which fall within the above expressed concept of the present invention are also intended as protected by the present application.

What is claimed is:

1. A protection device for the protection of the respiratory tract of a person comprising:
a mask body of one of rigid or semi-rigid, soft material adapted for covering a person's mouth and nose,
at least one pair of housings placed on sides of said mask body for holding a sleeve inside each housing,
a spring inserted inside each sleeve, each spring having a first end, and a second end with a fixing arrangement enabling said ends of the two springs to join together behind the head of the person wearing the device,
wherein each spring is surrounded substantially along a first portion fixed in the sleeve by a first cylinder of non-breathable fabric material and along a second portion by a second cylinder of one of breathable fabric material or other material adapted for filtering the air, so that the air introduced into at least one sleeve is captured from the back of the person's head,
wherein the front part of the mask body comprises a housing box removable from the mask body and including a fan which facilitates, by a connection hole, recirculation of the air inside the mask body, which is sucked by at least one of the two sleeves through a connection tube placed between the sleeves and the housing box, by a non-return valve placed in a lower end part of the respective sleeve.

2. A protection device according to claim 1, wherein said fan is placed inside a cylindrical body joined to the connection tube so as to better convey the incoming air into the mask body.

3. A protection device according to claim 1, wherein said housing box is equipped with a gasket in plastic material and is joined to the mask body by a snap closure.

4. A protection device according to claim 1, wherein said fan is energized by wireless rechargeable batteries and is controlled by an on/off button placed at the front of said housing box.

5. A protection device according to claim 1, wherein the second cylinder is made of a non-woven fabric or of a material of the type used for FFP1, FFP2 or FFP3 masks capable of filtering the air.

6. A protection device according to claim 1, wherein the springs are made of one of metal or plastic material.

7. A protection device according to claim 1, wherein the mask body is formed by a first layer and a second layer substantially superimposed on each other and communicating through at least one hole so as to guarantee the passage of the air between them.

8. A protection device according to claim 1, wherein each sleeve is provided with at least one pair of fins or another fixing arrangement so as to make it the sleeve removable from the housing.

9. A protection device according to claim 1, wherein the fixing arrangement of the second end of each spring includes a strip of Velcro fabric adapted for providing a hooking and release at the back of the head of the person wearing the device very quick.

10. A protection device according to claim 1, wherein the mask body is equipped with housings specially shaped to accommodate bluetooth headsets.

11. A protection device according to claim 1, wherein the at least one said sleeve is equipped, internally, in a central part, with a container for a pad, said container being formed by male and female parts overlapping each other and provided with lateral openings, in which the male part has an enlarged base in the shape of a truncated cone equipped with a housing seat for the first end of a respective spring, an appendix that fits inside a central part of the non-return valve and a support of the pad.

12. A protection device according to claim 11, wherein said appendix is provided, internally, with at least one protuberance adapted to fit into a corresponding cavity in an end part of the connection tube which allows the passage of air into the housing box of the fan and is equipped with an enlarged base in a shape of a truncated cone, which abuts against an external part of the non-return valve locking the connection tube in place.

13. A protection device according to claim 11, wherein said container is adapted to be closed or opened by rotation of the female part on relative to the male part and, in the closed position, walls of the female part close respective openings of the male part, while in the open position, respective openings of the male and female parts overlap each other, allowing the passage of the air circulating inside the sleeve.

14. A protection device according to claim 11, wherein the female part of the container is equipped, on an inner wall of the latter, with at least one tooth which fits inside an “L”-shaped groove on an outer wall of the male part so that, when it reaches the end of the vertical stroke and by rotation R of the female part, the latter stops and remains in position.

15. A protection device according to claim 13, in which the rotation movement of the female part on relative to the male part is facilitated by the presence of a hexagonal pin which fits into a specially shaped housing on a top of the female part, wherein a fixing element, is attached to said pin and is adapted for fixing said sleeve behind the person's head and has inside it a housing seat for an end part of the spring.

16. A protection device according to claim 11, wherein the filtered incoming air is adapted to reach, through said openings, the pad which is adapted to be soaked in an aromatic, balsamic or medicinal substance and is adapted to release the substance as it passes, so that aromatized/medicated air is conveyed inside the connection tube to the housing box of the fan and, therefore, inside the mask body.

17. A protection device according to claim 11, wherein said pad is activated by heating a resistance placed in the immediate vicinity and/or in contact with the pad and the resistance is adapted to be activated by an electro-thermal command placed in a front part of the housing box.

18. A protection device according to claim 1, wherein an air inlet into the mask body takes place from both sleeves and the respective non-return valves while an air outlet takes place via a non-return valve placed in a front of a housing box and a filter made of FFP1, FFP2 or FFP3 material.

19. A protection device according to claim 1, in which the cylinders are made of fabric of breathable material.
